(19) United States
(12) Patent Application Publication
(10) Pub. No.: US 2021/0196246 A1
(43) Pub. Date: Jul. 1, 2021

(54) Suction Based Saliva Treatment and Collector Device

(71) Applicant: Northeastern University, Boston, MA (US)

(72) Inventors: Ming WANG, Ipswich, MA (US); Sheyda NAZARIAN, Boston, MA (US); Yunqing DU, Quincy, MA (US)

(21) Appl. No.: 17/137,612

(22) Filed: Dec. 30, 2020

Related U.S. Application Data
(60) Provisional application No. 62/954,787, filed on Dec. 30, 2019.

(51) Int. Cl. A61B 10/00; A61B 5/00

(52) U.S. Cl. CPC A61B 10/0051; A61B 2217/005; A61B 5/4277

(57) ABSTRACT

A device for treatment and collection of saliva provides a base containing aa suction source. A tip having an inlet for collecting a saliva sample is removably attached to the base. A filter material, selected to allow passage of a target biomarker in the saliva sample, is disposed within the tip on a fluid path from the inlet. A collection chamber is disposed on the fluid path downstream of the filter material. The suction source is in fluid communication with the fluid path to draw saliva into the inlet of the tip, through the filter material, and to the collection chamber. Methods for collection of a saliva sample are also provided.

### CROSS REFERENCE TO RELATED APPLICATIONS

This application claims priority under 35 U.S.C. § 119(e) of U.S. Provisional Application No. 62/954,787, filed on 30 Dec. 2019, entitled “Suction Based Saliva Treatment and Collector Device,” the disclosure of which is hereby incorporated by reference.

### BACKGROUND

Whole saliva is a clear, slightly acidic (pH 6 to 7) mix of fluid secreted from minor and major salivary glands. Saliva is a very complex mixture, including more than 99% water, electrolytes, metabolites, hormones, and proteins such as mucins, immunoglobulins, and enzymes. Mucin is one of the most abundant proteins in saliva. In accord with its brush-like structure and negatively charged backbone, it introduces a viscoelastic property to saliva.

Saliva plays several crucial roles in maintaining oral health such as lubrication, antimicrobial activity, cleaning activity, dilution of sugars from food and drink, buffering, etc. It is also suitable for disease diagnosis and biomarker detection. For example, saliva has been used to detect HIV, hepatitis A, B and C virus, oral cancer, breast cancer, pancreatic cancer, lung cancer, cardiovascular disease, and diabetes. Therefore, saliva is a promising substitution for blood, serum, plasma, and urine as a diagnostic sample.

However, the lack of a standard method of collection and sample treatment is hindering its potential applications. The concentrations of most of the biomarkers in the saliva are much lower in comparison to their concentrations in blood, which limits the development of saliva-based point-of-care testing and laboratory-based devices for disease diagnostics. Also, while electrochemical methods of biomarker detection have many advantages including being simple, rapid and inexpensive, they have not been widely used with real biological samples. One of the aspects limiting the efficacy of electrochemical methods for biological samples such as saliva, is the interference of the numerous other proteins and biomarkers that cause the selectivity and specificity of the test to suffer.

There is a need for improved technology for rapid, inexpensive, and convenient collection of saliva samples for analysis of biomarkers and processing of saliva to render it suitable for analysis.

### SUMMARY

A saliva treatment and collector device and method are provided to collect saliva utilizing suction and filter the sample to remove undesired components and enable detection of different biomarkers present in saliva. In the saliva collection device, suction applies a force to produce a partial vacuum by the removal of air and lowering the pressure, which forces fluid through the filter material into a collection chamber. This device can be used in conjunction with a point-of-care biomarker detection system. The device can be used at home or in a laboratory without surveillance by trained personnel. The device can be used in veterinary clinics and laboratories.

Embodiments of a saliva treatment and collector device include a base containing a suction or low pressure source. A tip having an inlet for collecting a saliva sample is removably attached to the body. A filter material, selected to allow passage of a target biomarker in the saliva sample, is packaged within the tip on a fluid path from the inlet. A collection chamber is disposed on the fluid path downstream of the filter material. The suction source is in fluid communication with the fluid path to draw saliva into the inlet of the tip, through the filter material, and to the collection chamber.

Additional aspects and features of the technology described herein include the following:

1. A device for collection of saliva, comprising:

a base comprising a body, a suction source disposed within the body;

a tip removably attached to the base, the tip having a channel therethrough and an inlet to the channel;

a filter material supported by the tip on a fluid path from the inlet of the tip, the filter material selected to allow passage of a target biomarker in a saliva sample; and

a collection chamber disposed on the fluid path downstream of the filter material, the suction source in fluid communication with the fluid path to draw saliva through the inlet of the tip and through the filter material to the collection chamber.

2. The device of 1, further comprising a capsule mounted within the channel, the filter material disposed within the capsule, the capsule including a discharge outlet downstream of the filter material.
3. The device of any of 1-2, wherein the collection chamber is mounted to the capsule, and the discharge outlet extends within an upstream portion of the collection chamber.
4. The device of any of 1-3, wherein the collection chamber includes an aperture in the upstream portion for communication with a suction chamber within the body of the base.
5. The device of any of 1-4, wherein the collection chamber includes an aperture therein, and the base includes a suction chamber in the body, the suction source including a suction outlet disposed to draw air on the fluid path into the suction chamber, and to draw a saliva sample on the fluid path into the collection chamber.
6. The device of any of 1-5, wherein the collection chamber is attached to the tip and extends into the body, and the collection chamber is removable from the body with the tip to transfer a filtered sample in the collection chamber to a detector.
7. The device of any of 1-6, wherein the tip is removable such that the filter material can be inserted into a sensor apparatus to provide a direct contact of or force-induced transfer of the target biomarker.
8. The device of any of 1-7, wherein the suction source comprises a vacuum pump disposed within the body.
9. The device of any of 1-8, wherein the suction source provides a suction pressure selected to allow passage of the target biomarker through the filter material.
10. The device of any of 1-9, wherein the suction source is operative to apply a suction pressure of at least 5 inches of mercury.
11. The device of any of 1-10, wherein the suction source is operative to apply a suction pressure in the range of 5 to 25 inches of mercury.
12. The device of any of 1-11, wherein the filter material has one or more of a density and type of material selected to allow passage of the target biomarker, and/or the filter material is selected to reduce a viscosity of the saliva sample.
13. The device of any of 1-12, wherein the filter material has a density selected to allow passage of the target biomarker, and/or the filter material is selected to reduce a viscosity of the saliva sample, the density ranging from 5 mg/m³ to 200 mg/m³.
14. The device of any of 1-13, wherein the filter material is selected from the group consisting of cotton, cellulose, nylon, glass, polysulfone, carbon, polyester, aramid, boron, cellulose acetate, nitrocellulose, polytetrafluoroethylene (PTFE), silver, quartz, polypropylene, asbestos, polyurethane, acrylic, poly vinylidene fluoride (PVDF), nitrocellulose, and polyethersulfone (PES) and combinations thereof.
15. The device of any of 1-14, wherein the filter material is selected from the group consisting of cotton, silica, and nylon.
16. The device of any of 1-15, wherein the filter material is cotton.
17. The device of any of 1-16, wherein the filter material is cotton having a density ranging from 20 mg/m³ to 60 mg/m³, and the target biomarker is glucose.
18. The device of any of 1-17, wherein the filter material is a fiber, nanofiber, foam, sponge, or membrane.
19. The device of any of 1-18, wherein the filter material further includes a reagent or surfactant selected from the group consisting of salt solution containing sodium chloride, potassium sorbate, sodium benzoate, alkylphenol ethoxylate, alcohol ethoxylate, tergitol, teriton, propylene oxide, sodium polyoxyethylene lauryl ether, polyoxyethylene derivative, polyoxyethylene glycerol fatty acid ester, polypropylene glycol, alkoxylated glycerin, polyoxyethylene castor oil, alkyldiphenyloxide disulfonate salt, sodium lauryl sulfate, sodium alkyl naphthalene sulfonate, polyoxyethylene alkylether carboxylic acid, and polyoxyethylene alkylether carboxylates, and combinations thereof.
20. The device of any of 1-19, wherein the filter material is a material treated by an air plasma treatment or sterilized by an autoclave, ethylene oxide gas, gamma irradiation or ultraviolet irradiation.
21. The device of any of 1-20, wherein the filter material is selected to allow passage of the target biomarker selected from the group consisting of a hormone, cytokine, protein, enzyme, antibody, nucleic acid, antigen, virus marker, bacterium marker, fungus marker, drug, metabolite, electrolyte, inorganic substance, tumor marker, cell, particle, and lipid, and combinations thereof.
22. The device of 21, wherein:

the hormone is selected from the group consisting of cortisol, androgens, estriol, estrogen, progesterone, testosterone aldosterone, melatonin, dehydroepiandrosterone (DHEA), and insulin, and combinations thereof;

the cytokine is selected from the group consisting of interleukin, interleukin IK-1beta, interleukin IL-6, interleukin IL-8, tumor necrosis factor, and troponin and combinations thereof;

the protein or enzyme is selected from the group consisting of amylase, pepsin, matrix metalloproteinases, C-reactive protein (CRP), mucins, lactoferrin, and antimicrobial peptide, and combinations thereof;

the growth factor is selected from the group consisting of epidermal growth factor and vascular growth factor and combinations thereof;

the antibody or antigen is selected from the group consisting of immunoglobulin A, immunoglobulin G, immunoglobulin M, HIV antibody, and SARS-CoV-2 antibody, and combinations thereof;

the nucleic acid is selected from the group consisting of human and microbial DNA, mRNA, microRNA, and tRNA-derived small RNA (sRNA), and combinations thereof;

the virus marker is selected from the group consisting of a marker for SARS-CoV-2, marker for SARS-CoV-1, marker for HIV-1 and -2, marker for hepatitis A, B, and C, marker for flu, marker for HSV-1 and -2, marker for EBV, marker for HPV, marker for CMV VZV, marker for HCV, and marker for Ebola, and combinations thereof;

the bacterium maker a is selected from the group consisting of a marker for P. gingivalis, marker for S. mutans, marker for Lactobacillus spp., marker for T. forsythia, marker for E. coli, marker for H. pylori, and marker for M. tuberculosis, and combinations thereof;

the fungus marker is selected from the group consisting of a marker for candida and marker for aspergillus;

the drug is selected from the group consisting of an anticonvulsant, chemotherapeutic agent, antibody, antineoplastic agent, analgesic, drug of abuse, and ethanol, and combinations thereof;

the metabolite or electrolyte is selected from the group consisting of a phosphate, calcium, sodium, potassium, glucose, chloride, nitrate, uric acid, amino acids, lipids, and carbohydrates, and combinations thereof; or

the tumor marker is selected from the group consisting of CA 15-3, HER2/neu, CA 19-9, p53, leptin, CA 125, alpha-fetoprotein, CEA, somatic mutation in tumor suppressor genes, loss of heterozygosity, promoter hypermethylation of genes, and microsatellite DNA alteration, and combinations thereof.

23. The device of any of 1-22, wherein the collection chamber includes a reagent stored therein.
24. A method of detecting a target biomarker comprising:

collecting a saliva sample from a human subject or a non-human animal using the device of any of 1-23; and

detecting the target biomarker in the saliva sample.

25. The method of 24, wherein the target biomarker is selected from the group consisting of a hormone, cytokine, protein, enzyme, antibody, nucleic acid, antigen, virus marker, bacterium marker, fungus marker, drug, metabolite, electrolyte, inorganic substance, tumor marker, cell, particle, and lipid, and combinations thereof.
26. The method of any of 24-25, wherein the device is used in salivary based point-of-care testing, a laboratory, a veterinary clinic, or a subject's home.
27. The method of any of 24-26, wherein the device is used in a cancer test, pregnancy test, salivary ovulation test, infectious disease test, allergic disease test, glucose detection, HIV test, SARS-CoV-2 test, COVID-19 test, influenza virus test, Cushing's disease test, nicotine test, hypogonadism test, an immunoassay, salivary antibody detection test, DNA analysis, RNA analysis, or ELISA assay, polymerase chain reaction test, ancestry test, genetic fingerprinting test.
28. The method of any of 24-27, wherein the device is used to collect a salivary sample of a non-human animal, the animal selected from the group consisting of dog, cat, pig, gerbil, sheep, cow, goat, horse, snake, mouse, rat, bird, rabbit, raccoon, snake, and monkey.
29. The method of any of 24-28, further comprising removing the tip from the body and inserting the collection chamber into a sensor apparatus to provide a direct contact with or force-induced transfer of the saliva sample.
30. A kit comprising the device of any of 1-23 and a plurality of additional tips removably attachable to the body, each additional tip including a filter material therein, and instructions for use thereof.
31. The kit of 30, wherein the filter material in each of the additional tips has one or more of a density and type of material selected to allow passage of the target biomarker or a different biomarker, and/or is selected to reduce a viscosity of the saliva sample.
32. The kit of any of 30-31, further comprising one or more reagents for detection, quantification, or analysis of the target biomolecule.
33. A system for electrochemical detection and/or quantification of glucose, comprising:

the device of any of 1-23, wherein the device is configured for detection and/or quantification of glucose; and

a glucose sensor.

34. The system of 33, wherein the glucose sensor is an electrochemical sensor.

### DESCRIPTION OF THE DRAWINGS

FIG. 1A is a front view of a removable tip of a saliva collection and treatment device.

FIG. 1B is a cross-sectional view along line A-A of FIG. 1A of the tip of the saliva collection and treatment device.

FIG. 2A is a front view of a saliva collection and treatment device.

FIGS. 2B and 2C are the cross-sectional views along line B-B of FIG. 2A of the saliva collection and treatment device.

FIG. 3A is a front view of the saliva collection and treatment device of FIGS. 2A-2C in conjunction with the tip of FIGS. 1A-1B.

FIG. 3B is a cross-sectional view along line C-C of FIG. 3A of the saliva collection and treatment device in conjunction with the tip.

FIG. 4 shows viscosity measurement test results, in which viscosity of filtered saliva is compared with an unfiltered sample and buffer solution as a baseline. Three types of filter material at five different density or compression states have been used in this test. 100% viscosity reduction means the viscosity of the filtered sample is the same as the buffer solution as a baseline.

FIG. 5 shows total protein reduction percentage of each filtered saliva sample using BCA assay kit, in which the total protein concentration of the filtered saliva sample is compared with the unfiltered sample. Three types of filter material at five different density or compression states have been used in this test.

FIG. 6 shows glucose content measurement test results, in which the glucose concentration of the filtered saliva sample is compared with the unfiltered sample. Three types of filter material at five different density or compression states have been used in this test.

### DETAILED DESCRIPTION

The technology described herein provides for the rapid and convenient collection of a saliva sample that is processed for analysis by filtering out any large interfering components while leaving the target biomarker, and lowering the sample's viscosity. This can be realized by using a filter material selected for each desired biomarker, as described herein. The filtration system described herein can therefore play a role in bridging laboratory and point-of-care systems with respect to a saliva-based detection technology. The present technology can enable the use of electrochemical processes to measure target biomarkers from saliva, for example, in a patient's own home.

The technology provides a whole saliva treatment and collector device that utilizes suction as a collection technique, and a filter material and filter treatment procedure to make collected saliva samples more pure by filtering out any large interfering components. Suction applies a force to produce a partial vacuum by the removal of air and lowering the pressure which further forces fluid into a vacant space within a collection chamber. The device can be used at home or in a laboratory without surveillance by trained personnel.

Embodiments of a saliva treatment and collector device include a base including a body containing a suction or low pressure source. A tip having an inlet for collecting a saliva sample is removably attached to the body of the base. A filter material, selected to allow passage of a target biomarker in the saliva sample, is packaged within the tip on a fluid path from the inlet. A collection chamber is disposed on the fluid path downstream of the filter material. The suction source is in fluid communication with the fluid path to draw saliva into the inlet of the tip, through the filter material, and to the collection chamber.

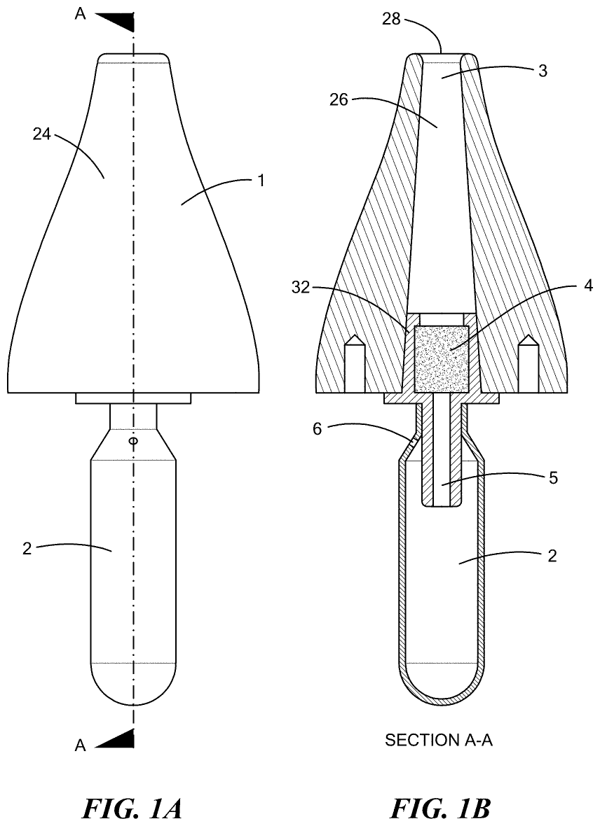

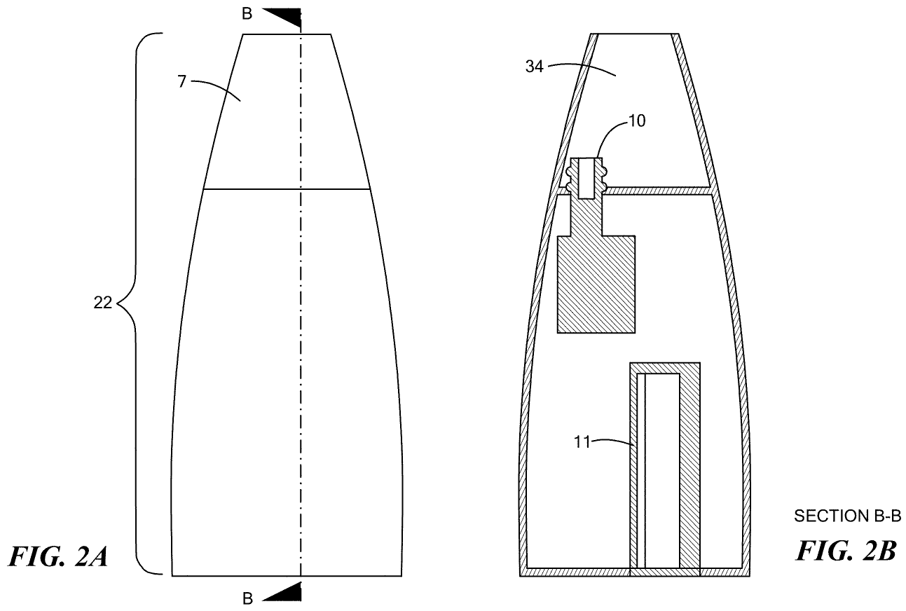

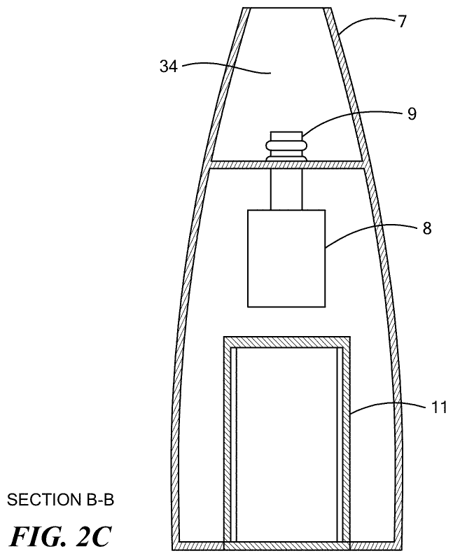

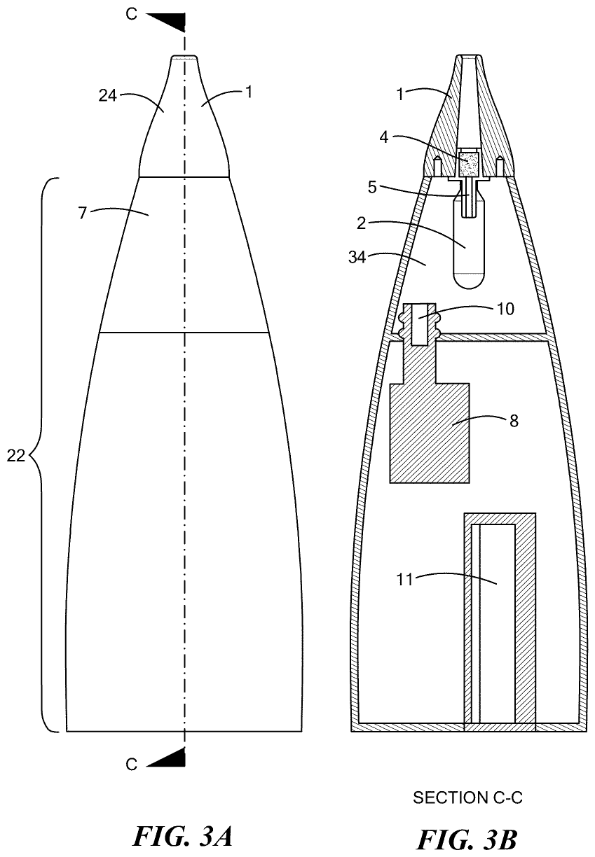

More particularly, referring to the embodiment illustrated in FIGS. 1A to 3B, a treatment and collector device can include two parts, a base 7, comprising a body 22 that can be a reusable or permanent part, and a removable tip 1, which can be a disposable part. The tip is the part that contacts the sample during collection. The tip includes a housing 24 that attaches to the body in any suitable manner to be removable while sealable to the body to maintain a sufficiently low pressure within the device to draw a saliva sample therethrough. The housing of the tip includes a channel 26 therethrough from an inlet 28 at one end through which saliva can be drawn under suction. The housing can be tapered such that the inlet is sufficiently narrow to ease placement in a patient's mouth while still allowing for collection of a saliva sample.

A filter material 4 is placed within or adjacent a downstream end of the channel. The filter material can be placed within a capsule 32 or other holder that fits within the channel of the tip. The volume of the capsule can range, for example, from 500 to 800 mm³. The capsule can include a discharge outlet 5 that extends below the housing of the tip to guide a saliva sample into a collection chamber 2. An upstream end of the collection chamber can be attached to the discharge outlet in any suitable manner. An aperture or opening 6 is provided near the upstream end in the collection chamber, above the bottom of the discharge outlet, through which suction pressure can be applied to the interior of the collection chamber and to the channel to draw the sample into the interior of the collection chamber.

A suction or low pressure source 8 such as vacuum pump is provided within the body 7 to apply a desired suction pressure through a suction outlet 10. In the embodiment illustrated, the body includes a suction chamber 34 in a sealed upper region 36 of the body in fluid communication with the suction outlet 10 of the pump and the aperture 6 into the collection chamber. The vacuum pump can be sealed to the body in any suitable manner, for example, using an O-ring 9 surrounding the suction outlet 10. The vacuum pump can draw saliva in the mouth on a fluid flow path through the inlet of the channel in the tip 1, through the filter material 4 and the discharge outlet 5 into the collection chamber 2.

It will be appreciated that any suitable pump to draw saliva on a fluid path into and through the filter material can be used. The pump can be electrically operated and can be powered in any suitable manner, such as by one or more batteries (not shown), which can be housed in a battery compartment 11 within the body 7. The compartment can include a removable cover to access the batteries for replacement. Alternatively or in addition, the body can include an electrical outlet for attachment to a power source via a power cord for operation and/or to charge the one or more batteries.

A suitable suction pressure can be selected to be comfortable and convenient for patients, while also being high enough to collect and filter a sufficient volume of saliva sample. In some applications, the suction pressure provided by the pump can range from 5 to 25 inches of mercury, depending on the type and density of the filter material. In some applications, the suction pressure can be at least 5 inches of mercury. In some applications, the suction pressure can be less than 5 inches of mercury or greater than 25 inches of mercury. The suction pressure can also be selected in conjunction with the density of the filter material.

The collection chamber 2 can be detached from the body 7 along with the tip 1 for application of the saliva sample to a sensor or sensor apparatus, any further measurement site, or for further transport to a laboratory. In some embodiments, the collection chamber with the saliva sample can be removed from the tip as needed to apply the saliva sample to a sensor or sensor apparatus, any further measurement site, or transported to a laboratory. The tip, the filter material, and the collection chamber can be disposed of after each use of the device, minimizing or eliminating any contamination interfering with the test upon further use of the body of the device. The filter material is packaged with the tip, and consequently, new, clean filter material can be used each time the device is used by placing a new tip 1 on the body 7. The body in which the vacuum pump is housed can be used repeatedly without being contaminated with the saliva sample.

The tip 1 can be sealed to the body 7 in any suitable manner, for example, by making the tip out of a soft or compressible material (e.g. silicon) and/or providing an O-ring or other sealing mechanism to ensure a sufficiently low pressure with minimal air leakage between the tip and the body to draw a saliva sample through the tip into the collection chamber. The body can be made of any suitable material, such as durable hard plastic. One or more of the tip, body, filter capsule, and collection chamber can be formed in any suitable manner from any suitable material, such as by machining, injection molding, overmolding, casting, or by one of several rapid manufacturing methods, such as stereolithography, fused deposition, three-dimensional printing, or selective sintering. The tip can be removably attached to the body in any suitable manner, such as, without limitation, a friction fit, snap fit, screw threads, or bayonet mount.

The filter type, filter material, density of the filter material, and any additive and/or treatment procedure can be specifically selected for each target biomarker, depending on what molecules are desired to be filtered. The filtered saliva can have a desired property depending on the target biomarker. For the case of small molecule detection, the concentration of larger components such as proteins can be reduced. For example, for glucose detection, the filter material and its density can be selected to remove salivary mucous, lower its viscosity and remove large molecules (compared to glucose) while not altering the concentration of glucose of the original sample. In some embodiments, a filter type and/or density of the filter material can be selected to provide retention on the filter of particles having a size in the range of 2 nm to 100 nm, such as 2 nm, 3 nm, 4 nm, 5 nm, 6 nm, 7 nm, 8 nm, 9 nm, 10 nm, 12 nm, 15 nm, 20 nm, 25 nm, 30 nm, 40 nm, 50 nm, 60 nm, 70 nm, 80 nm, 90 nm, or 100 nm, thereby allowing biomarker molecules of smaller molecular size to pass through the filter and be detected. For example, glucose has a molecular size of about 1 nm and could pass through a filter having a particle size cutoff of 5 nm or greater.

Any suitable type of filter can be used. For example, depth filters, such as foams, fibers, or fabrics, membrane filters, or a combination of multiple filter types, can be used. The filter type used in the device, the density of the filter material, any additives and/or any treatment can be selected based on the target biomarker. The filtration can be based, for example, on molecular size or chemical bonds (e.g., covalent bonding, ionic bonding, hydrogen bonding, hydrophobic bonding, or van der Waals interaction) between the filter material and molecules present in the sample.

The filter material can be selected from the group consisting of cotton, cellulose, nylon, glass, polysulfone, carbon, polyester, aramid (e.g., Kevlar®), boron, cellulose acetate, nitrocellulose, polytetrafluoroethylene (PTFE), silver, quartz, polypropylene, asbestos, polyurethane, acrylic, poly vinylidene fluoride (PVDF), nitrocellulose, and polyethersulfone (PES) and combinations thereof.

The filter material can be provided in a variety of forms, such as fiber, nanofiber, foam, sponge, or porous membrane form. For example, the filter material can be a cotton fiber, cellulose fiber, nylon fiber, glass fiber, polysulfone fiber, carbon fiber, polyester fiber, aramid fiber (e.g., Kevlar®), boron fiber, cellulose acetate fiber, nitrocellulose fiber, polytetrafluoroethylene (PTFE) fiber, quartz fiber, polypropylene fiber, asbestos fiber, or acrylic fiber, or combinations thereof. The filter material can be a polyurethane foam. The filter material can be a nylon membrane, PTFE membrane, PVDF membrane, nitrocellulose membrane, PES membrane, ion exchange membrane, mixed cellulose esters membrane, cellulose acetate membrane, cellulose nitrate membrane, polypropylene membrane, glass fiber membrane, silver membrane, or cotton membrane, or combinations thereof.

The density of the filter material in particular can affect properties of the filtered sample such as viscosity, total protein concentration, and target molecule concentration. In some embodiments, the density of the filter material can range from 5 mg/m³ to 200 mg/m³. In some embodiments using silica fiber, the density can range from 10 mg/m³ to 200 mg/m³. For glucose biomarker detection, silica fiber with a density range of 60 mg/m³ to 130 mg/m³ is suitable. In some embodiments using cotton fiber, the density can range from 10 mg/m³ to 150 mg/m³. For glucose detection, cotton fiber with a density range of 20 mg/m³ to 60 mg/m³ is suitable. In some embodiments using nylon fiber, the density can range from 10 mg/m³ to 150 mg/m³. For the case of bacteria, antibody, protein and virus detection, a nylon fiber filter with density range of 5 to 50 mg/m³, a silica fiber filter with density range of 50 to 200 mg/m³, or a cotton fiber filter with density of 5 to 40 mg/m³ are generally suitable for salivary sample filtration. It will be appreciated however, that the density range can be different for each biomarker, and suitable density ranges can be readily determined for any desired biomarker based on the technology described herein.

In order to introduce a desired property (such as affinity for an interfering molecule, hydrophobicity or hydrophilicity) to the filtering material, additional filter treatments can be performed to filter out interfering molecules. Treatments can include addition of one or more surfactants or reagents. The collection chamber can also include a reagent stored therein. The reagent can be, for example, an antimicrobial agent, protein stabilizer, protease inhibitor, or sodium azide, or a combination thereof. The filter material can be a material treated by an air plasma treatment or sterilized by an autoclave, ethylene oxide gas, gamma irradiation, electron beam irradiation, or ultraviolet irradiation.

A reagent or surfactant can be selected from the group consisting of salt solution containing sodium chloride, potassium sorbate, sodium benzoate, alkylphenol ethoxylate, alcohol ethoxylate, tergitol, teriton, propylene oxide, sodium polyoxyethylene lauryl ether, polyoxyethylene derivative, polyoxyethylene glycerol fatty acid ester, polypropylene glycol, alkoxylated glycerin, polyoxyethylene castor oil, alkyldiphenyloxide disulfonate salt, sodium lauryl sulfate, sodium alkyl naphthalene sulfonate, polyoxyethylene alkylether carboxylic acid, and polyoxyethylene alkylether carboxylates, and combinations thereof.

The filter material can be selected to allow passage of a biomarker selected from the group consisting of a hormone, cytokine, protein, enzyme, antibody, nucleic acid, antigen, virus marker, bacterium marker, fungus marker, drug, metabolite, electrolyte, inorganic substance, tumor marker, cell, particle, and lipid, and combinations thereof.

For example, hormones can include, without limitation, cortisol, androgens, estriol, estrogen, progesterone, testosterone aldosterone, melatonin, dehydroepiandrosterone (DHEA), and insulin.

Cytokines can include, without limitation, interleukins (IK-1beta, IL-6, IL-8), tumor necrosis factor, and troponin.

Proteins and enzymes can include, without limitation, amylase, pepsin, matrix metalloproteinases, C-reactive protein (CRP), mucins, lactoferrin, and antimicrobial peptides.

Growth factors can include, without limitation, epidermal growth factor and vascular endothelial growth factor.

Antibodies and antigens can include, without limitation, immunoglobulin A, immunoglobulin G, immunoglobulin M, HIV antibody, and SARS-CoV-2 antibody.

Nucleic acids can include, without limitation, human and microbial DNA, mRNA, microRNA, and tRNA-derived small RNA (sRNA).

Virus markers can include any molecular component characteristic of a virus, including a protein, nucleic acid, or antigen, and including markers for, without limitation, SARS-CoV-2, SARS-CoV-1, HIV-1 and HIV-2, hepatitis A, B, and C viruses, influenza virus, HSV-1 and HSV-2, EBV, HPV, CMV, VZV, and Ebola virus.

Bacterium markers can include any molecular component characteristic of a bacterium, including a protein, nucleic acid, or antigen, and including, without limitation, markers for P. gingivalis, S. mutans, Lactobacillus spp., T. forsythia, E. coli, H. pylori, and M. tuberculosis.

Fungus markers can include any molecular component characteristic of a fungus, including a protein, nucleic acid, or antigen, and including, without limitation, markers for candida and aspergillus.

Drugs can include, without limitation, anticonvulsants, chemotherapeutic agents (including antibodies and antineoplastic agents), analgesics, drugs of abuse, and ethanol.

Metabolites and electrolytes can include, without limitation, phosphate, calcium, sodium, potassium, glucose, chloride, nitrate, uric acid, amino acids, lipids, and carbohydrates.

Tumor markers can include, without limitation, CA 15-3, HER2/neu, CA 19-9, p53, leptin, CA 125, alpha-fetoprotein, CEA, somatic mutations in tumor suppressor genes, and microsatellite DNA alterations.

The device can be used in association with a variety of saliva-based tests, such as a cancer test, pregnancy test, ovulation test, infectious disease test, allergic disease test, glucose detection, human immunodeficiency virus (HIV) test, rubella virus test, SARS-CoV-2 test, COVID-19 test, influenza virus test, hepatitis A, B, and C, Cushing's disease test, nicotine test, hypogonadism test, an immunoassay, antibody detection test, DNA analysis, RNA analysis, or ELISA assay, polymerase chain reaction test, ancestry test, or genetic fingerprinting test.

The device can be used to collect saliva samples for the detection of biomarkers for a virus, antibodies against a virus, and biomarkers of an immune response to a virus, including viruses such as SARS-CoV-2, hepatitis A, B, and C, human immunodeficiency virus, and rubella virus.

The device can be used in association with a variety of assays for biomarkers, such as reverse transcription polymerase chain reaction (RT-PCR). The device can be used, for example, in association with saliva-based assays for the detection of hormones, cytokines, proteins, enzymes, antibodies, nucleic acids, antigens, viruses markers, bacteria markers, fungi markers, drugs, metabolites, electrolytes, inorganic substances, tumor markers, cells, particles, and lipids.

The device can be used with human subjects. The technology can be used in saliva-based point-of-care tests for health monitoring and disease diagnosis, in saliva-based bio-sensing systems for home use, in saliva-based laboratory tests to improve sensitivity and the detection limit of measurements, in conjunction with salivary electrochemical detection tests, and with elders and infants to collect their saliva sample.

The device can be used a veterinary office or clinic. The device can be used to collect saliva samples from non-human animals, such as, without limitation, a canine, feline, equine, bovine, swine, rodent, or primate. The device can be used to collect saliva samples from non-human animals including the group consisting of dog, cat, pig, gerbil, sheep, cow, goat, horse, snake, mouse, rat, bird, rabbit, raccoon, and monkey.

The technology includes a kit having a saliva treatment and collector device as described herein and instructions for its use. The kit can include a body and one or a plurality of tips. The tips can include the same or a variety of filter materials. Tips can be packed individually with a filter material and subsequently sterilized for inclusion in a kit.

The technology includes a system for the electrochemical detection of a biomarker. The system can include the device as described herein and an electrochemical sensor for the detection of the target biomarker.

The technology includes a system for the electrochemical detection of glucose. The system can include the device as described herein and an electrochemical glucose sensor. Suitable glucose sensors are described in WO 2014/110492 and WO 2018/107168, incorporated herein by reference.

Embodiments of a saliva treatment and collection device as described herein can be used in a variety of methods. Methods can include gathering a filtered sample into a collection chamber for further transfer and measurements. Parts of the device that come into contact with the sample, e.g., the tip, can be disposable and can be discarded after each test to assure the accuracy and safety of the method. The sample can be directly inserted into another sensor or sensor apparatus and/or combined with a sensor apparatus through direct contact or force-induced transfer to a targeted sensor.

The technology described herein can provide several features and advantages. The filter materials used in the device, selected to be specific for each biomarker, can accurately filter out any interfering molecules from the sample and lower its viscosity, which can result in improving the sensitivity and detection limit of the tests. The filtration mechanism, filter material and its weight, density and/or treatment procedure enable filtering out large interfering components from a saliva sample. An appropriate filter material can be used for each desired biomarker. The filtration system can play a role in bridging laboratory and point-of-care systems with respect to a saliva-based detection. Having a disposable piece makes it easier for everyday use without the need for washing it. Having an interchangeable filter embedded in the disposable tip makes it possible to reuse the same base for various target molecule detection systems or for repeated tests. This technology can deliver filtered saliva sample to any detection site such as a point-of-care sensor or laboratory test. The technology can enable the use of electrochemical processes to measure target biomarkers from saliva in a subject's home.

The device can minimize the risk of transmission of contact with saliva droplets or particles or aerosols. The ease of sample collection enables the device to be used in conjunction with disease diagnosis, monitoring of response to treatment, assessment of disease severity and progression of a disease.

The device and method can overcome disadvantages of the use of spitting and cotton swabs to collect saliva samples. For example, when a cotton swab is used to collect a saliva sample, the amount of cotton material used is much higher compared to using cotton as a filter in conjunction with the suction collector device and method as described herein. The difference between the cotton filter used in the collector device described herein and the cotton swab collection method is the lower weight of cotton used in the present collector device, which can subsequently provide more quantitative recovery of biomarkers and improve the accuracy of biomarker detection and/or quantification.

Also, the use of cotton swabs results in the collection of stimulated whole saliva, which has been shown to introduce undesired bias even though it is among the most common methods of collection. Stimulation of saliva secretion affects the properties of the saliva in ways such as changing the bacterial profile, flow rate, and total protein concentration. Spitting is a method that can be used to collect unstimulated saliva, but it is not the most elegant or convenient for everyday usage and it can be messy, which discourages saliva donors.

In the work done by Michishige (Michishige, Fumiko, et al. “Effect of saliva collection method on the concentration of protein components in saliva.” The journal of medical investigation 53.1, 2 (2006): 140-146) the properties of saliva collected by suction, spitting and the swab methods were compared. A saliva ejector and an external aspirator were utilized to collect saliva by suction. It was concluded that the concentration of total protein, S-IgA, trypsin-like activity, and human airway trypsin-like protease were the same in saliva collected by suction and spitting but remarkably lower in the saliva sample collected by cotton swab, which it was considered the least reliable method of collection. Therefore, the suction method is as reliable as spitting in terms of sample purity, uniformity, and integrity preservation. The device described herein collects unstimulated whole saliva, particularly since the external tip does not stay in the mouth during secretion of saliva, and avoids localized salivary gland secretion without introducing any bias, while it provides compliance and facilitates saliva collection in comparison to the spitting method. Consequently, suction is the collection method used herein, because it allows for unstimulated saliva collection. In the aforementioned study, a saliva sample was frozen at −80° C. after collection, which resulted in precipitation of mucin and particulate matters; in other similar studies, collected saliva samples needed to be centrifuged to remove mucin and particulate matter. In contrast, in the device described herein, the combination of a suction collection mechanism with determined pressures, selected filter material or combination of filters with determined density, weight, additives (such as surfactants), and treatments (such as air plasma) can result in collecting a purer and less viscous sample such that no further sample treatment (e.g. freezing, boiling, centrifugation, or the like) is needed.

Suction-based saliva ejectors and aspirators are used in dentistry as stationary devices to evacuate the oral cavity of extra fluid. Generally, such dental devices consist of a saliva ejector which is a plastic tube connected to a stationary vacuum coupling unit on the dental chair to apply suction. The main goal of using this device is to remove the excess saliva, but not to use the saliva once it has been extracted; subsequently, no filtration or other treatment takes place in this device. This device is a large stationary device which cannot be used with a home testing system as a portable saliva collector.

The Carlson-Crittenden saliva collector (also known as a Lashley cup) consists of an inner and an outer chamber housed in a plastic cup. The inner chamber is connected to plastic tubing that carries saliva and the outer chamber is attached to a suction inducing device. By applying suction to the outer part, the device is fixed to the cheek and held in place. Saliva freely drains to the inner chamber and the connected tube. This device collects saliva secreted by the parotid gland, and it needs to be placed over the main parotid excretory duct by a skilled person and cannot be used to collect the whole saliva. The applied vacuum is only responsible for holding the device in place and not to collect saliva. As with saliva ejectors, this device cannot be used to collect the whole saliva in conjunction with point-of-care or laboratory-based testing systems.

Generally, suction applies a force to produce a partial vacuum by the removal of air and lowering the pressure which further forces fluid into a vacant space. Suction has been used in devices such as baby nasal aspirators and breast pumps and many more applications. In the baby nasal aspirator, suction is utilized to draw nasal mucus from the nasal passage to improve breathing. In a breast pump, suction is used to extract milk from the breasts. Suction is used in many applications, but there are limited electrical suction-based whole saliva collector systems that can be used for point-of-care or laboratory-based testing. The present device is a portable whole saliva collector and treatment device that utilizes electrical suction as a collection method, which can be used at home or in the laboratory without surveillance by trained personnel.

With regard to the spitting method of collection, saliva is accumulated on the floor of the mouth and the donor spits the gathered saliva in a storage cup. Some examples of the spitting method of saliva collection are, U.S. Pat. No. 9,113,850, SalivaBio's Passive Drool collector by Salimetrics, LLC, SD-3000 saliva collection kit by Samplify Bio Inc. Orange, ONE ON-500 kit by DNA Genoteck Co. All these devices consist of a closed chamber to store the collected sample that are designed differently in each case. A saliva donor expectorates into a closed chamber until the desired volume is collected; the collected sample is unfiltered whole saliva. This method of saliva collection is not elegant, could be a messy action for everyday usage, some users consider it rude to spit, and it cannot be used for infants. It could also be troublesome for elderly users and subjects suffering from the effects of a stroke. Also, using this collection method, saliva cannot freely pass through a filter.

Examples of cotton swab collection methods are described in US Pat. Pub. No. 2006/0036206 (application Ser. No. 10/535,813), U.S. Pat. Nos. 7,618,591, 9,198,641 by Oasis Diagnostic, Co. SE-2030 saliva collection kit by Samplify Bio Inc., U.S. Pat. No. 8,287,809, SalivaBio's Oral Swab (SOS) by Salimetrics LLC. In these examples, saliva is collected by masticating an absorbent material such as dental cotton, and further squeezing or centrifuging the absorbent material to get the collected saliva sample. In ORAcollect.Dx by DNAgenoTek, a sponge is used to absorb saliva upon which the saliva is released into a stabilizer solution without any filtration occurring. This device is intended to be used in human DNA laboratory tests only.

It has been reported that cotton swab collection method is compatible with some biomarker detection systems such as HIV antibodies, DHEA-S, and cotinine, while it is not compatible with some other biomarker detection systems such as glutathione, testosterone, DHEA, progesterone, estradiol, SIgA, cortisol, myoglobin, melatonin (especially at lower levels) since a cotton swab introduces error to the analysis and could result in a false measurement. This phenomenon might be either because of imperfect recovery of target molecules from the cotton swab, due to absorption to the cotton, where in this case polystyrene might be the better choice for them, or the presence of an interfering substance in cotton and further releasing to the saliva sample is causing the false measurement. Afterward, the nonreversible trapping of the aforementioned biomarkers reduces the reliability and accuracy of the absorbent swab collection method. The absorbent material used in this collection method limits the range of biomarkers that could be detected from saliva. Hence, the cotton swab method is not advantageous.

The suction method of saliva collection used herein does not suffer from the problem of inelegance in spitting, and it does not introduce bias as does the cotton swab collection method. It does not alter the original properties of saliva, and it can easily be used with elders and infants. Hence, the suction method of saliva collection is advantageous when compared to the other two methods. The saliva collection and treatment device described herein collects and purifies a saliva sample by utilizing a suction pump with a suitable suction pressure, combined with a suitable filtering material or combination of multiple filters with density, weight, additives, and treatments selected to detect one or multiple biomarkers.

In summary, the device provides a saliva treatment system which can be used for salivary biomarker detection. Because most salivary biomarker concentrations are significantly lower compared to blood, this device and method can remove interfering molecules, lower the viscosity and improve sensitivity and detection range of salivary based point-of-care and laboratory-based tests. This treatment can be realized in the saliva collector device which can facilitate the collection and treatment of the saliva sample.

### Example

A saliva collector device as described with reference to FIGS. 1A-3B was fabricated. Experimental results demonstrated that the saliva collector device collected saliva from a mouth of a patient in about 10 seconds, and during collection, saliva sample passed through the filter material in the tip.

Three depth filters, using silica (glass), cotton and nylon fibers, were used for the case of glucose detection. A capsule filter with a defined filter volume reservoir was 3D-printed and different weights of filter material were embedded in the reservoir to provide a random tortuous path for sample to pass through and get filtered. Several different compression states were used for each depth filter material: a silica fiber density range of 10-200 mg/m³, a nylon fiber density range of 10 to 150 mg/m³, and a cotton filter density range of 10 to 150 mg/m³.

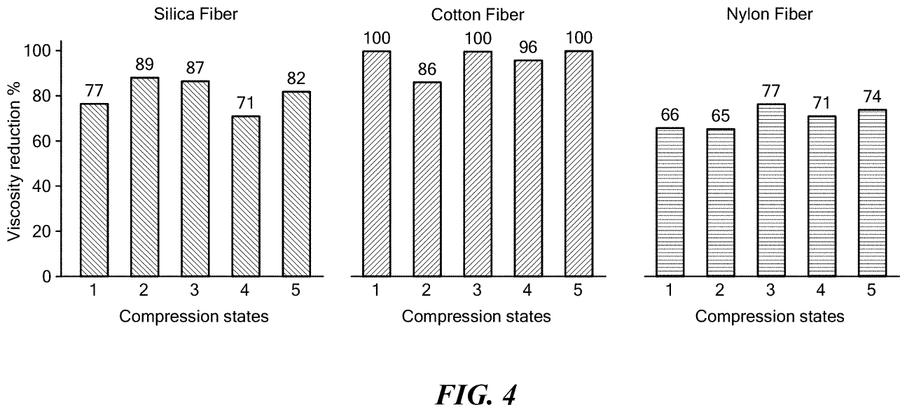

Compression states as shown in FIGS. 4-6 are as follows:

| | | Density (w/v; g/m³) |
|---|---|---|
| Filter Material | Compression States | ×10³ |
| Nylon | 1 | 10-35 |
| | 2 | 35-45 |
| | 3 | 45-50 |
| | 4 | 50-60 |
| | 5 | 60-75 |
| Glass (Silica) | 1 | 50-80 |
| | 2 | 80-100 |
| | 3 | 100-125 |
| | 4 | 125-140 |
| | 5 | 140-160 |
| Cotton | 1 | 10-35 |
| | 2 | 35-50 |
| | 3 | 50-65 |
| | 4 | 65-80 |
| | 5 | 80-100 |

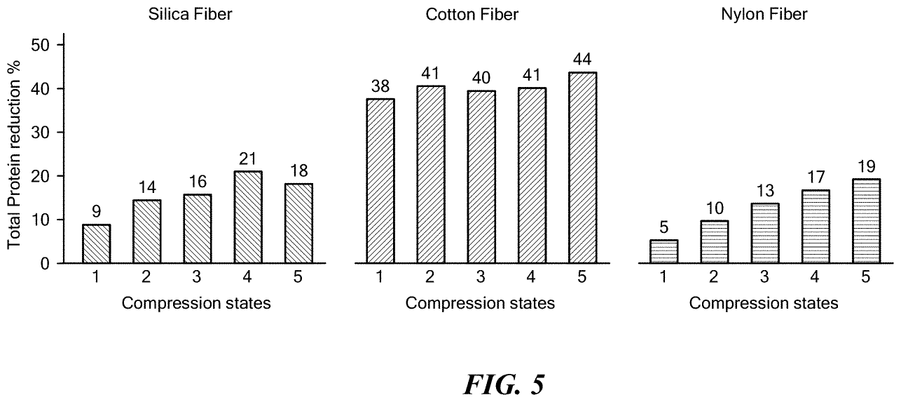

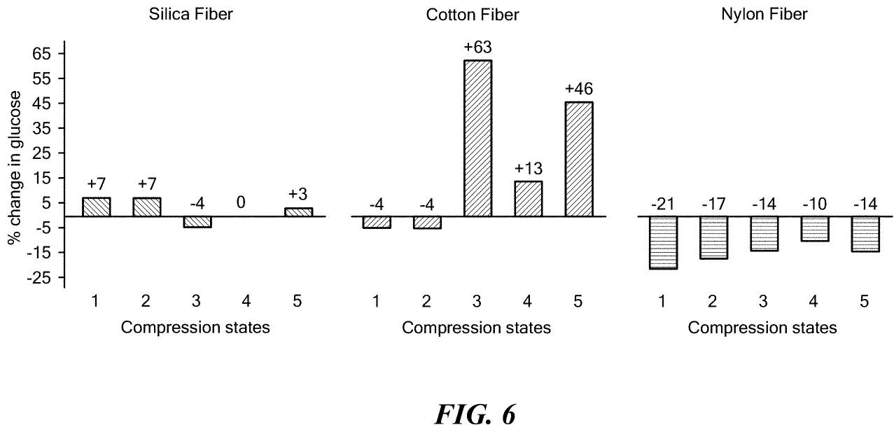

Samples collected by the device were tested in conjunction with a glucose detection system. In this case, it was required to filter out salivary mucous and lower the viscosity of the sample. It was shown that the viscosity of the sample could be markedly reduced (measured by microVISC™ viscometer by RHEOSENSE, INC) shown in FIG. 4, in which the viscosity of the filtered saliva was compared with unfiltered sample and buffer solution as a baseline. The larger molecules compared to glucose were preferably removed, leaving behind a purer sample. To do so, total protein concentration of filtered and unfiltered saliva was measured by a BCA Protein Assay Kit (by Thermo Scientific), and it was shown that total protein concentration could be reduced by 40% shown in FIG. 5. Simultaneously, the original concentration of glucose remained unchanged. This property was investigated by measuring glucose concentration of filtered and unfiltered saliva sample by glucose colorimetric/fluorometric assay kit (by BioVision, Inc), shown in FIG. 6. Accordingly, it was concluded that the cotton filter with compression state number 2 performed as desired.

As used herein, “consisting essentially of” allows the inclusion of materials or steps that do not materially affect the basic and novel characteristics of the claim. Any recitation herein of the term “comprising,” particularly in a description of components of a composition or in a description of elements of a device, can be exchanged with “consisting essentially of” or “consisting of.”

It is to be understood that the technology is not limited to the exact details of construction, operation, exact materials or embodiments or aspects shown and described, and that various modifications, substitution of equivalents, alterations to the compositions, and other changes to the embodiments and aspects disclosed herein will be apparent to one of skill in the art.

What is claimed is:

1. A device for collection of saliva, comprising:
a base comprising a body, a suction source disposed within the body;
a tip removably attached to the base, the tip having a channel therethrough and an inlet to the channel;
a filter material supported by the tip on a fluid path from the inlet of the tip, the filter material selected to allow passage of a target biomarker in a saliva sample; and
a collection chamber disposed on the fluid path downstream of the filter material, the suction source in fluid communication with the fluid path to draw saliva through the inlet of the tip and through the filter material to the collection chamber.

2. The device of claim 1, further comprising a capsule mounted within the channel, the filter material disposed within the capsule, the capsule including a discharge outlet downstream of the filter material.

3. The device of claim 2, wherein the collection chamber is mounted to the capsule, and the discharge outlet extends within an upstream portion of the collection chamber.

4. The device of claim 1, wherein the collection chamber includes an aperture in the upstream portion for communication with a suction chamber within the body of the base, and the base includes a suction chamber in the body, the suction source including a suction outlet disposed to draw air on the fluid path into the suction chamber, and to draw a saliva sample on the fluid path into the collection chamber.

5. The device of claim 1, wherein the collection chamber is attached to the tip and extends into the body, and the collection chamber is removable from the body with the tip to transfer a filtered sample in the collection chamber to a detector.

6. The device of claim 1, wherein the suction source comprises a vacuum pump disposed within the body.

7. The device of claim 1, wherein the suction source provides a suction pressure selected to allow passage of the target biomarker through the filter material, wherein the suction pressure is at least 5 inches of mercury.

8. The device of claim 1, wherein the suction source is operative to apply a suction pressure in the range of 5 to 25 inches of mercury.

9. The device of claim 1, wherein the filter material has one or more of a density and type of material selected to allow passage of the target biomarker, and/or the filter material is selected to reduce a viscosity of the saliva sample.

10. The device of claim 1, wherein the filter material has a density selected to allow passage of the target biomarker, and/or the filter material is selected to reduce a viscosity of the saliva sample, the density ranging from 5 mg/m³ to 200 mg/m³.

11. The device of claim 1, wherein the filter material is selected from the group consisting of cotton, cellulose, nylon, glass, polysulfone, carbon, polyester, aramid, boron, cellulose acetate, nitrocellulose, polytetrafluoroethylene (PTFE), silver, quartz, polypropylene, asbestos, polyurethane, acrylic, poly vinylidene fluoride (PVDF), nitrocellulose, and polyethersulfone (PES) and combinations thereof.

12. The device of claim 1, wherein the filter material is selected from the group consisting of cotton, silica, and nylon.

13. The device of claim 1, wherein the filter material is cotton having a density ranging from 20 mg/m³ to 60 mg/m³, and the target biomarker is glucose.

14. The device of claim 1, wherein the filter material is a fiber, nanofiber, foam, sponge, or membrane.

15. The device of claim 1, wherein the filter material further includes a reagent or surfactant selected from the group consisting of salt solution containing sodium chloride, potassium sorbate, sodium benzoate, alkylphenol ethoxylate, alcohol ethoxylate, tergitol, teriton, propylene oxide, sodium polyoxyethylene lauryl ether, polyoxyethylene derivative, polyoxyethylene glycerol fatty acid ester, polypropylene glycol, alkoxylated glycerin, polyoxyethylene castor oil, alkyldiphenyloxide disulfonate salt, sodium lauryl sulfate, sodium alkyl naphthalene sulfonate, polyoxyethylene alkyl ether carboxylic acid, and polyoxyethylene alkyl ether carboxylates, and combinations thereof.

16. The device of claim 1, wherein the filter material is a material treated by an air plasma treatment or sterilized by an autoclave, ethylene oxide gas, gamma irradiation or ultraviolet irradiation.

17. The device of claim 1, wherein the filter material is selected to allow passage of the target biomarker selected from the group consisting of a hormone, cytokine, protein, enzyme, antibody, nucleic acid, antigen, virus marker, bacterium marker, fungus marker, drug, metabolite, electrolyte, inorganic substance, tumor marker, cell, particle, and lipid, and combinations thereof.

18. The device of claim 1, wherein the filter material is cotton.

19. A method of detecting a target biomarker comprising:
collecting a saliva sample from a human subject or a non-human animal using the device of claim 1; and
detecting the target biomarker in the saliva sample.

20. A kit comprising the device of claim 1 and a plurality of additional tips removably attachable to the body, each additional tip including a filter material therein, and instructions for use thereof, and wherein the filter material in each of the additional tips has one or more of a density and type of material selected to allow passage of the target biomarker or a different biomarker, and/or is selected to reduce a viscosity of the saliva sample.

21. A system for electrochemical detection and/or quantification of glucose, comprising:
the device of claim 1, wherein the device is configured for detection and/or quantification of glucose; and
a glucose sensor.
